(19) United States
(12) Patent Application Publication
(10) Pub. No.: US 2021/0000890 A1
(43) Pub. Date: Jan. 7, 2021

(54) COMPOSITIONS AND METHODS FOR DENTAL CARE

(71) Applicants: The State of Israel, Ministry of Agriculture & Rural Development, Agricultural Research Organization, Rishon-LeZion (IL); Yissum Research Development Company of the Hebrew University of Jerusalem Ltd., Jerusalem (IL)

(72) Inventors: Moshe SHEMESH, ModiIn (IL); Doron STEINBERG, Jerusalem (IL)

(73) Assignees: The State of Israel, Ministry of Agriculture & Rural Development, Agricultural Research Organization, Rishon-LeZion (IL); Yissum Research Development Company of the Hebrew University of Jerusalem Ltd., Jerusalem (IL)

(21) Appl. No.: 16/919,161

(22) Filed: Jul. 2, 2020

Related U.S. Application Data
(60) Provisional application No. 62/869,586, filed on Jul. 2, 2019.

(51) Int. Cl. A61K 35/742; A61K 31/047; A61Q 11/00; A61C 15/02; A61C 15/04

(52) U.S. Cl. CPC A61K 35/742; A61K 31/047; A61C 15/041; A61C 15/02; A61Q 11/00

(57) ABSTRACT

A composition comprising at least one sugar alcohol and a population of viable probiotic bacteria belonging to the class Bacilli is disclosed. The product is devoid of sugar or comprises no more than 5% of the amount of said sugar alcohol in the composition, the composition being formulated for oral delivery.

### RELATED APPLICATION(S)

This application claims the benefit of priority under 35 USC § 119(e) of U.S. Provisional Patent Application No. 62/869,586 filed on Jul. 2, 2019, the contents of which are all incorporated by reference as if fully set forth herein in their entirety.

### FIELD AND BACKGROUND OF THE INVENTION

The present invention, in some embodiments thereof, relates to probiotic compositions for dental care, and more particularly to compositions comprising probiotic bacteria belonging to the genus Bacillus and at least one sugar alcohol.

Bacillus subtilis is a Gram-positive, non-pathogenic, spore and biofilm-forming bacterium which ubiquitously exists in the different ecological niches. Being basically a soil bacterium, B. subtilis successfully colonises a plant root mainly due to its metabolic diversity in utilizing different carbohydrate sources. To control osmoregulation, many plants produce polyhydric alcohols such as sorbitol and mannitol. Therefore, the ability to metabolise those sugars may provide a big advantage to root colonizing bacteria.

Bacillus species have been used as probiotics for at least 50 years, but scientific interest in characterizing their probiotic potential has massively occurred in the last 15 years. B. subtilis possesses two major advantages as a probiotic bacterium. First, B. subtilis has been found in in-vitro and in-vivo assessments to be safe in the food supplements⁸. Second, its ability to form dormant and highly resistant endospores. These advantages enable keeping this bacterium in its dormant state though various environmental stresses without any deleterious effect on its viability.

Probiotics have been associated mostly with the health of gastrointestinal tract (GIT), however, recent studies suggest that probiotic bacteria could also be beneficial for oral health¹¹⁻¹³. The administration of probiotic bacteria for the oral cavity to achieve the probiotic effect can be during food consumption for long or short period^{14,15}, tablets¹⁶ chewing gums^{17,18} and in mouth rinsing¹⁹. It was however established that the most prevalent oral disorder—dental caries, which is caused mainly due to oral biofilm-forming bacteria, also highly depends on the consumed diet²⁰. One of the most cariogenic bacterium in the oral cavity is Streptococcus mutans²¹. S. mutans can bind to the pellicle using adhesion-like proteins. After initial adhesion, S. mutans secretes enzymes producing extracellular polysaccharide, which is considered important for further bacterial adhesion and acceleration of biofilm formation^{20,22}. Accumulation of S. mutans as a biofilm is the result of the bacteria's self-adhesion mechanisms, but is also highly dependent on dietary components. The correlation between an abundance of sucrose, biofilm formation and caries has been well documented in the literature. Sucrose increases biofilm biomass, since it serves as a substrate for extracellular polysaccharide (EPS) production²³, and its fermentation by cariogenic bacteria generates organic acids²⁴. One of the approaches to control biofilm formation and the development of caries is a replacement of sucrose with alcoholic sugars such as sorbitol or mannitol²⁵. However, the reduction in caries rates are still insufficient^{26,27}.

Additional background art includes US Patent Application No. 20190022153 and US Patent Application No. 201903 88485.

### SUMMARY OF THE INVENTION

According to an aspect of the present invention, there is provided a composition comprising at least one sugar alcohol and a population of viable probiotic bacteria belonging to the class Bacilli, wherein the product is devoid of sugar or comprises no more than 5% of the amount of the sugar alcohol in the composition, the composition being formulated for oral delivery.

According to an aspect of the present invention, there is provided a food comprising the composition described herein.

According to an aspect of the present invention, there is provided a device which is coated with the composition described herein.

According to an aspect of the present invention, there is provided a method of preventing or treating dental caries in a subject comprising administering to the subject a therapeutically effective amount of a population of viable probiotic bacteria belonging to the class Bacillus and at least one sugar alcohol, thereby preventing or treating dental caries.

According to an aspect of the present invention, there is provided a method of preventing or treating dental caries in a subject comprising applying the composition or device described herein to the oral cavity of a subject in need thereof, thereby preventing or treating dental caries in the subject.

According to embodiments of the present invention, the probiotic bacteria belong to the genus Bacillus.

According to embodiments of the present invention, the composition is a dental product.

According to embodiments of the present invention, the device is a toothpick or a dental floss.

According to embodiments of the present invention, the composition is selected from the group consisting of a wash, a paste, a chewing gum and an ointment. According to embodiments of the present invention, the food further comprises an artificial sweetener.

According to embodiments of the present invention, the probiotic bacteria belong to the species Bacillus subtilus.

According to embodiments of the present invention, the sugar alcohol is a six carbon sugar alcohol.

According to embodiments of the present invention, the six carbon sugar alcohol is selected from the group consisting of mannitol, sorbitol, galactitol, flucitol and iditol.

According to embodiments of the present invention, the sugar alcohol is mannitol or sorbitol.

According to embodiments of the present invention, the probiotic bacteria belong to the genus Bacillus.

According to embodiments of the present invention, the viable probiotic bacteria and the at least one sugar alcohol are co-formulated in a single composition.

According to embodiments of the present invention, the viable probiotic bacteria and the at least one sugar alcohol are formulated in separate compositions. According to embodiments of the present invention, the probiotic bacteria belong to the species Bacillus subtilus.

According to embodiments of the present invention, the sugar alcohol is a six carbon sugar alcohol.

According to embodiments of the present invention, the six carbon sugar alcohol is selected from the group consisting of mannitol, sorbitol, galactitol, flucitol and iditol.

According to embodiments of the present invention, the sugar alcohol is mannitol or sorbitol.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1. B. subtilis mitigate biofilm formation by S. mutans in the presence of sorbitol and mannitol. S. mutans cells were grown with or without B. subtilis in TY medium supplemented with different concentrations of sorbitol or mannitol. TY medium alone and TY with 2% sucrose served as controls. The bacteria were incubated at 37° C. in 95% air/5% CO₂ for 24 h. For biofilm biomass quantification the formed biofilms were washed twice using PBS and stained with Crystal Violet staining. The data are displayed as biofilm biomass percentage compare to S. mutans biofilm that formed in TY with 2% sucrose. As opposed to sucrose, in all the concentration of sorbitol or mannitol, there was a significant reduction in the formed biofilm when both of the bacteria were grown together compare to S. mutans alone. The data is the mean and SD of data from at least three independent experiments, each performed in triplicate.*P value<0.01 compared to S. mutans biofilm alone in the same concentration of sugar.

FIGS. 2A-C. B. subtilis and S. mutans growth curves in the presence of sorbitol and mannitol. The bacteria were incubated at 37° C. 16 h. O.D (595 nnm) measurements were taken every 1 hour.

A. S. mutans or B. subtilis cells were grown in TY medium supplemented with different concentrations of sorbitol, TY medium alone served as control.

B. S. mutans or B. subtilis cells were grown in TY medium supplemented with different concentrations of mannitol, TY medium alone served as control.

While B. subtilis cells entered immediately into the log stage for all sugar concentrations, S. mutans had a long lag phase of approximately 3-4 hours and entered the stationary phase much earlier.

The data are the mean values of two independent experiments, each formed in duplicate.

C. B. subtilis and S. mutans cells were grown in M9 minimal medium or M9 supplemented with 0.2% Glucose, 50 mM sorbitol or 50 mM mannitol. While B. subtilis cells entered immediately into the log stage for all sugar concentrations, S. mutans had a long lag phase of approximately 3-4 hours when glucose was added to the media, while it did not grow in the presence of sorbitol or mannitol during the 16 h of growth.

The data are the mean values of two independent experiments, each formed in duplicate.

FIG. 3. Relative expression of gutB and mltD genes during bacterial growth in the presence of sorbitol and mannitol accordingly.

B. subtilis or S. mutans cells were grown in TY medium with or without 50 mM sorbitol or mannitol. For each time point a sample was taken for RNA extraction and relative gene expression using real-time RT-PCR.

In both of the bacteria, the genes were up-regulated in the presence of the sugars, whereas in B. subtilis the up-regulation was lower compare to the up-regulation in S. mutans.

The expression results represent mean±SD of two independent experiments.

FIGS. 4A-C. Relative expression of gutB and mltD genes during bacterial growth in the presence of sorbitol and mannitol accordingly.

B. subtilis or S. mutans cells were grown in TY medium with or without 50 mM sorbitol or mannitol. In each time point a sample was taken for RNA extraction and relative gene expression using real-time RT-PCR.

In both of the bacteria the genes were up-regulated in the presence of the sugars, whereas in B. subtilis the gene up-regulation was lower compared to the up-regulation in S. mutans.

The expression results represent mean±SD of two independent experiments.

FIGS. 5A-B. Key enzymes in sorbitol or mannitol metabolism are required for mitigating S. mutans biofilm.

S. mutans cells were grown with or without B. subtilis mutant cells (ΔgutB—FIG. 5A or ΔmltD—FIG. 5B) in TY medium supplemented with different concentrations of sorbitol or mannitol. TY medium alone and TY with 2% sucrose served as controls. The bacteria were incubated at 37° C. in 95% air/5% CO₂ for 24 h. For biofilm biomass quantification, the formed biofilms were washed twice using PBS and stained with Crystal Violet staining. The data are displayed as biofilm biomass percentage compare to S. mutans biofilm that formed in TY with 2% sucrose.

In both cases, B. subtilis mutant strains did not have a significant effect on the biofilm biomass in the related tested sugar, compare to S. mutans biofilm.

The data is the means and SD of data from at least three independent biological experiments, each performed in triplicate.*P value<0.05 compared to S. mutans biofilm alone in the same concentration of sugar.

FIGS. 6A-C. B. subtilis WT and mutant strains growth curves in M9 minimal medium in the presence of sorbitol and mannitol

B. subtilis cells were grown in M9 minimal medium supplemented with 0.2% Glucose (A) 50 mM sorbitol (B) or mannitol (C). The bacteria were incubated at 37° C. 16 h. O.D (595 nnm) measurements were taken every 1 hour.

B. subtilis ΔgutB cells were capable of growing on 50 mM mannitol as the WT strain but could not grow on 50 mM sorbitol as a sole carbon source. However, B. subtilis ΔmltD cells could not grow on 50 mM mannitol and also had a long lag phase (approximately 10 hours) before they started to grow on sorbitol as a sole carbon source. Importantly, all strain had a similar growth curve when glucose was the only carbon source.

The data are the mean values of two independent experiments, each formed in duplicate.

FIG. 7. Phenotypic characterization of S. mutans or S. mutans and B. subtilis dual-species biofilms using Crystal Violet staining. The staining strength is an indication of the amount of biofilm mass formed in TY medium supplemented with different concentrations of sorbitol or mannitol. TY medium alone and TY with 2% sucrose served as controls.

FIG. 8. CFU counts of B. subtilis or S. mutans in M9 medium with 50 mM sorbitol that were taken parallel to the bacterial respiration assay.

FIGS. 9A-B. B. subtilis mutant strains growth curves in the presence of sorbitol and mannitol;

B. subtilis cells were grown in TY medium supplemented with different concentrations of sorbitol (A) or mannitol (B). The bacteria were incubated at 37° C. 16 h. O.D (595 nnm) measurements were taken every 1 hour;

B. subtilis ΔgutB cells were capable of growing in TY with or without different concentrations of sorbitol or mannitol. However, B. subtilis ΔmltD cells were less able to grow in TY with or without different concentrations of sorbitol or mannitol;

The data are the mean values of two independent experiments, each formed in duplicate.

FIGS. 10A-B. S. mutans cells were grown with B. subtilis cells in TY medium supplemented with different concentrations of sorbitol or mannitol. TY medium alone and TY with 2% sucrose served as controls. The bacteria were incubated at 37° C. in 95% air/5% CO₂ for 24 h. DNA extraction was conducted using NaOH and real-time qPCR using species specific 16S rRNA primers was performed to quantified the amount of each bacterium (A. represent the amount of S. mutans DNA quantification and B. represent the amount of B. subtilis DNA quantification). The data display as mean±SD of 2 biological repeats, each performed in duplicate.

### DESCRIPTION OF SPECIFIC EMBODIMENTS OF THE INVENTION

The present invention, in some embodiments thereof, relates to probiotic compositions for dental care, and more particularly to compositions comprising probiotic bacteria belonging to the genus Bacillus and at least one sugar alcohol.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Bacillus subtilis is a Gram-positive probiotic bacterium that successfully colonizes plant roots due to its ability to utilize various sugars. The vast probiotic potential of B. subtilis has been recently demonstrated in numerous host organisms under different environmental conditions.

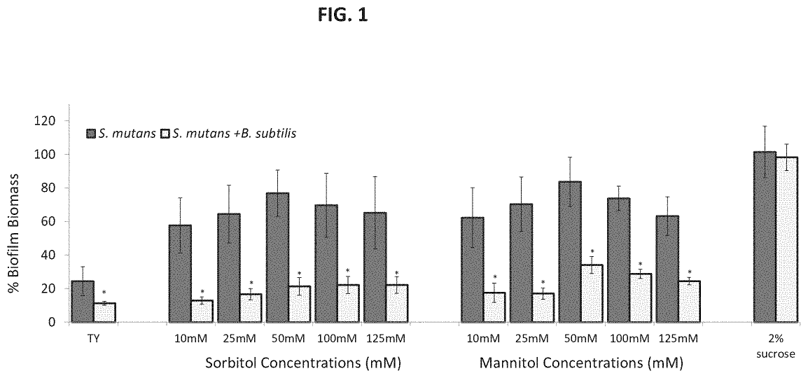

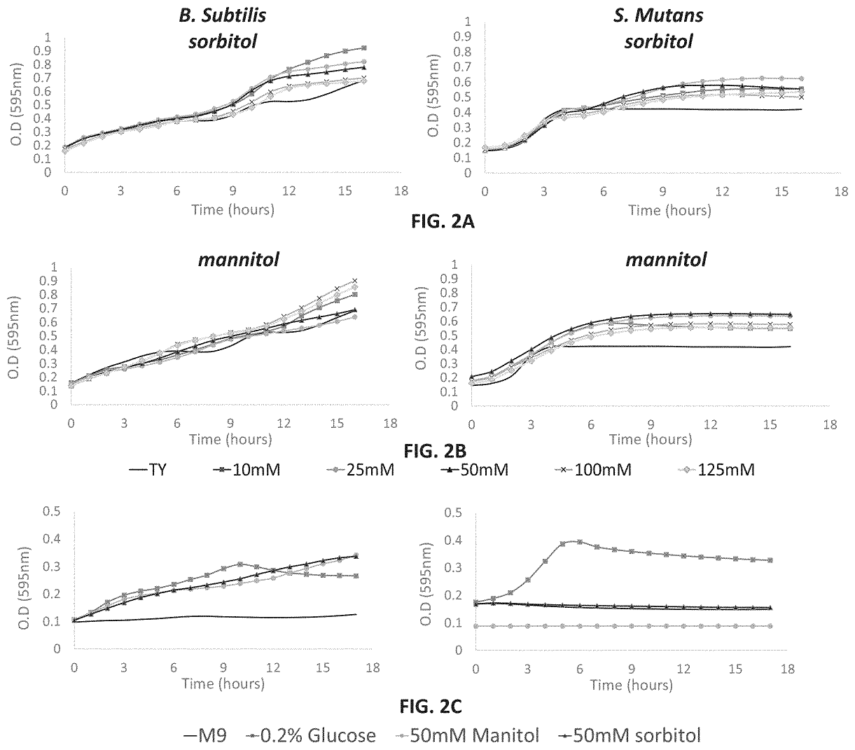

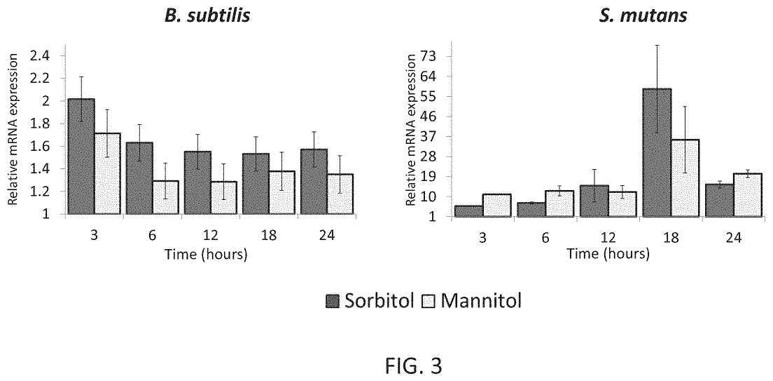

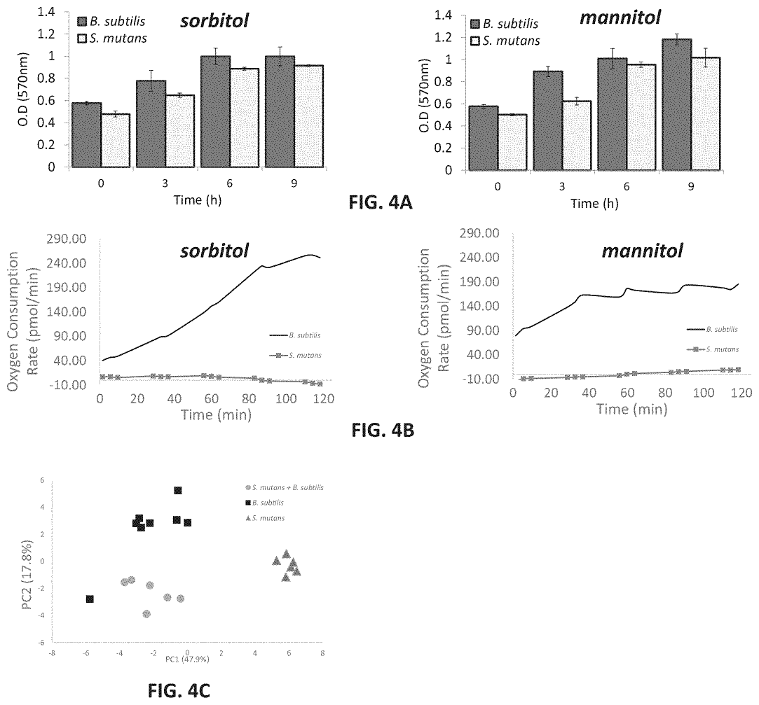

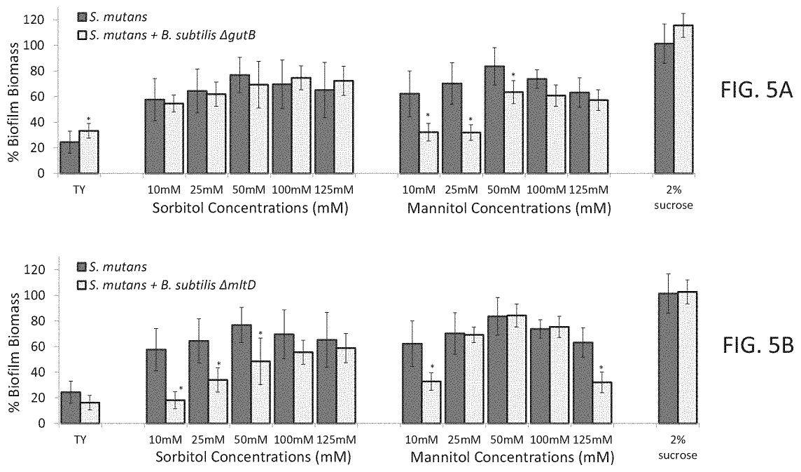

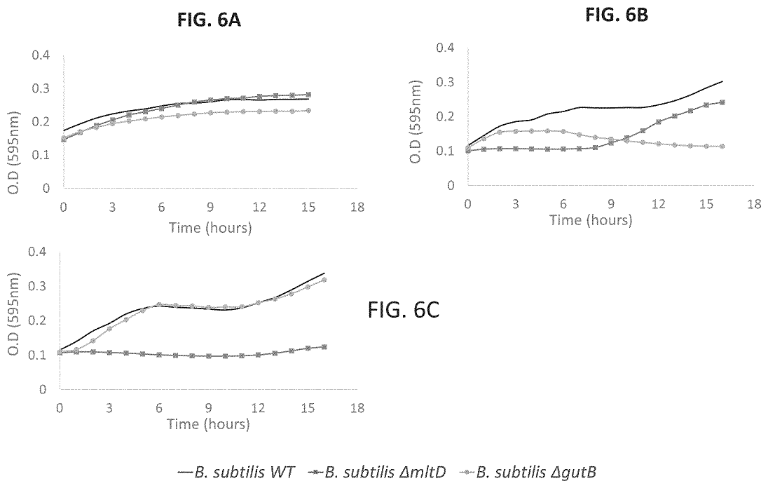

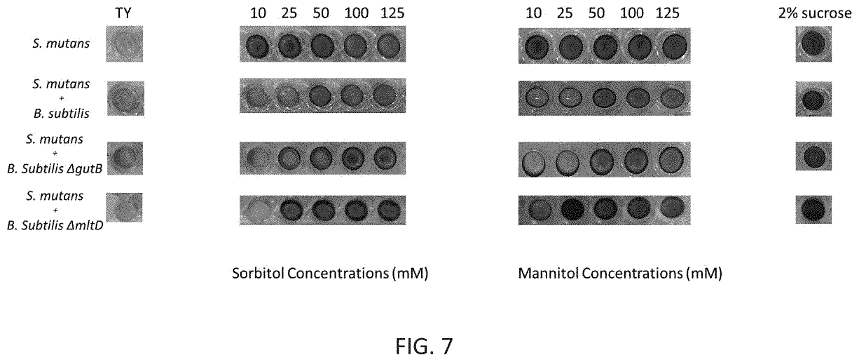

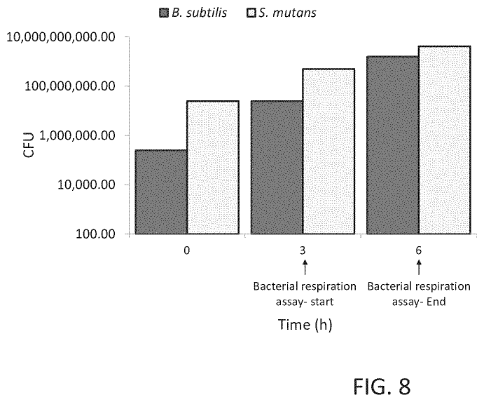

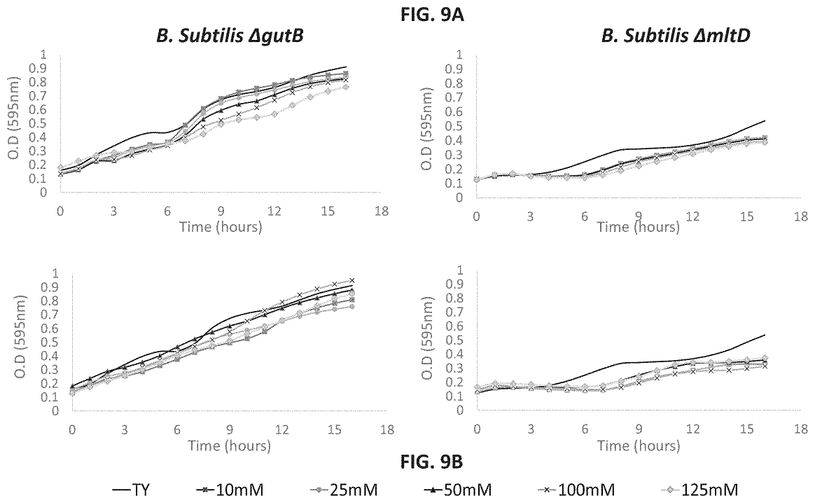

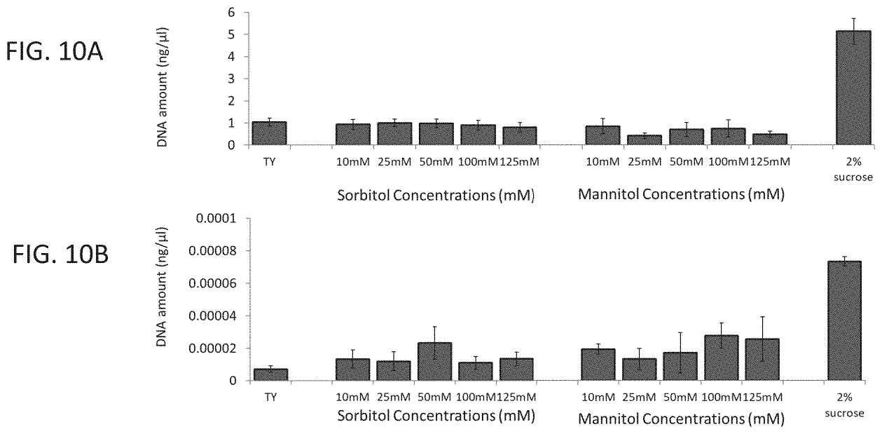

The present inventors have now examined the probiotic potential of B. subtilis against the pathogenic bacterium Streptococcus mutans, which is involved in various oral disorders due to its robust biofilm-forming capability. As seen in FIGS. 1 and 2A-C, B. subtilis cells attenuated biofilm formation by S. mutans during their dual growth in the presence of sugar alcohols. Transcription of genes encoding key enzymes in the metabolism of sugar alcohols by B. subtilis were highly induced (FIGS. 3 and 4A-C). Moreover, growth-curve analysis suggested that B. subtilis is more efficient at early utilizing sugar alcohols than S. mutans, as supported by the bacterial metabolic activity rates. Similarly, a comparison of secondary metabolites of mono and mixed cultures of B. subtilis and S. mutans indicated that B. subtilis is more active metabolically in the dual culture.

Finally, knock-out mutations of the genes encoding key enzymes in the central metabolic pathway significantly reduced B. subtilis' ability to mitigate biofilm formation by S. mutans (FIGS. 5A-B). The present inventors thus conclude that effective metabolism of sugar alcohols by B. subtilis reinforces the probiotic potential of this bacterium against pathogenic species such as S. mutans.

Consequently, the present teachings suggest that probiotic compositions comprising sugar alcohols and B. subtilis may be useful in the treatment and prevention of dental caries.

Thus, according to a first aspect of the present invention there is provided a method of preventing or treating dental caries in a subject comprising administering to the subject a therapeutically effective amount of a population of viable probiotic bacteria belonging to the class Bacillus and at least one sugar alcohol, thereby preventing or treating dental caries.

The colonization of the oral mucosa by disease-associated bacteria (e.g. Streptococcus nutans) and the formation of plaque can tip the microbial balance in the oral cavity towards an accumulation of detrimental microorganisms, which is also referred to as dysbiosis. Therefore, the microorganisms for use in the prevention and/or treatment of dental caries advantageously aides to avoid oral dysbiosis by balancing the mouth flora towards a healthy state.

The term “dental caries” refers to damage to a tooth that can happen when decay-causing bacteria in your mouth make acids that attack the tooth's surface, or enamel. This can lead to a small hole in a tooth, referred to herein as a cavity. If tooth decay is not treated, it can cause pain, infection, and even tooth loss.

Since dental caries and more specifically dental plaque is responsible for inflammatory conditions of the gums, the present inventors propose that the method disclosed herein may also be used to treat/prevent any disease associated with oral cavity inflammation including but not limited to gingivitis and also periodontitis.

The term “probiotic bacteria” as used herein refers to live bacteria which when administered in adequate amounts confer a health benefit on the host (e.g. human).

In one embodiment, the probiotic bacteria are of the class Bacilli, which includes the orders Bacillales and Lactobacillales.

Preferably, the bacteria of this aspect of the present invention are viable and are capable of forming a biofilm in the oral cavity of the subject.

Furthermore, the bacteria of this aspect of the present invention may also release anti-inflammatory agents and/or reduce the amount of pro-inflammatory agents in the oral cavity. For example, the bacteria of this aspect of the present invention may reduce the amount of any one of the following pro-inflammatory agents in the oral cavity: interleukin 1-beta (IL-1-beta), interleukin 6 (IL-6), interleukin 8 (IL-8), tumor necrosis factor alpha (TNFalpha), prostaglandin E2 (PGE2), 8-isoprostane, matrix metallopeptidase 9 (MMP9), 8-isoprostane and NFkappaB.

In one embodiment, the probiotic bacteria are of the genus Bacillus, e.g. species B. subtilis.

Exemplary strains of Bacillus species contemplated by the present invention include, but are not limited to B. paralicheniformis MS303, B. licheniformis MS310, B. paralicheniformis S127, B. subtilis MS1577, NCIB3610, B. subtilis natto, B. subtilis 168, B. subtilis PY79.

Other additional strains contemplated by the present invention include, the Lactobacillus paracasei LPc-G110 which has been deposited under the Budapest Treaty at the China Center for Type Culture Collection (CCTCC), Wuhan University, Wuhan 430072, China, under the accession number CCTCC M 2013691 by BioGrowing Co., Ltd., No. 10666 Songze Rd., Qingpu Shanghai 201700, China, on 23 Dec. 2013, and the strain Lactobacillus plantarum GOS 42 which has been deposited under the Budapest Treaty at the Leibniz Institut Deutsche Sammlung fur Mirkoorganismen and Zellkulturen GmbH (DSMZ), Inhoffenstr. 7B, 38124 Braunschweig, Germany, by Probi AB under the accession number DSM 32131 on 2 Sep. 2015. In addition, the probiotic bacteria may include the strain Lactobacillus delbrueckii subsp. lactis LL-G41 which has been deposited under the Budapest Treaty at the China Center for Type Culture Collection (CCTCC), Wuhan University, Wuhan 430072, China under the accession number CCTCC M 2016652 by BioGrowing Co., Ltd., No. 10666 Songze Rd., Qingpu Shanghai 201700, China, on 17 Nov. 2016, or the strain Lactobacillus plantarum Heal19, which has been deposited under the Budapest Treaty at the Leibniz Institut Deutsche Sammlung fur Mirkoorganismen and Zellkulturen GmbH (DSMZ) Inhoffenstr. 7B, 38124 Braunschweig, Germany, under the accession number DSM 15313 by Probi AB on 27 Nov. 2002 or the strain Lactobacillus paracasei NS9 is publicly available at the National Collection of Industrial, Food and Marine Bacteria, UK, (NCIMB), Ferguson Building, Craibstone Estate, Bucksburn, Aberdeen AB21 9YA, United Kingdom, under the accession number NCIMB 8823 (date of accession 1 Oct. 1956, deposited by University of Birmingham).

In one embodiment, a single strain of bacilli is administered. In another embodiment, at least two different strains of bacilli are administered. In another embodiment, at least three different strains of Bacilli are administered. In another embodiment, at least four different strains of Bacilli are administered. In another embodiment, at least five different strains of Bacilli are administered. In another embodiment, at least six different strains of Bacilli are administered. In another embodiment, at least seven different strains of Bacilli are administered. In another embodiment, at least eight different strains of Bacilli are administered. In another embodiment, at least nine different strains of Bacilli are administered. In another embodiment, at least ten different strains of Bacilli are administered.

In another embodiment, no more than 5 different strains of bacilli are administered. In another embodiment, no more than 10 different strains of bacilli are administered. In another embodiment, no more than 15 different strains of bacilli are administered. In another embodiment, no more than 20 different strains of bacilli are administered. As mentioned, the method of this aspect of the present invention contemplates administering the probiotic bacteria together with an alcohol sugar.

The sugar alcohol may be co-formulated with the probiotic bacteria in a single composition or each may be administered as separate components.

Thus, according to another aspect of the present invention there is provided a composition comprising at least one sugar alcohol and a population of viable probiotic bacteria belonging to the class Bacilli, wherein the product is devoid of sugar or comprises no more than 5% of the amount of said sugar alcohol in the composition, the composition being formulated for oral delivery.

Exemplary sugar alcohols include, but are not limited to ethylene glycol, glycerol, erythritol, threitol, arabitol, xylitol, ribitol, mannitol, sorbitol, galactitol, fucitol, iditol, inositol, volemitol, isomalt, malitol, lacitol, maltotriitol, maltotetraitol and polyglycitol. Particular sugar alcohols include sorbitol, mannitol and xylitol.

The amount of sugar alcohols provided is such that the amount of streptococcus bacteria in the oral cavity is reduced by as much as 10%, 20%, 30%, 40%, 50%, 60%, 70%, 80%, 90% or even 100%. For example, the concentration of sugar alcohol in the composition may be between 10-125 mM.

In one embodiment, the amount of sugar comprised in the compositions of the present invention is less than 5% the amount of sugar alcohols that are present.

In one embodiment, the amount of sugar comprised in the compositions of the present invention is less than 4% the amount of sugar alcohols that are present.

In one embodiment, the amount of sugar comprised in the compositions of the present invention is less than 3% the amount of sugar alcohols that are present.

In one embodiment, the amount of sugar comprised in the compositions of the present invention is less than 2% the amount of sugar alcohols that are present.

In one embodiment, the amount of sugar comprised in the compositions of the present invention is less than 1% the amount of sugar alcohols that are present.

The amount of sugar in the composition is preferably such that it prevents or reduces the amount of S. nutans bacteria in the oral cavity of a subject.

Exemplary sugars contemplated by the present invention include, but are not limited to sucrose, glucose, and fructose.

In another embodiment, the compositions are devoid of sugars.

In still another embodiment, the compositions comprise only trace amounts of sugars.

In still another embodiment, the compositions of the present invention are devoid of antimicrobial agents.

The bacteria is preferably provided in a composition in the range from 0.1 to 50%, most preferably in the range from 1 to 10%, in each case with respect to the total weight of the composition, and/or wherein the total amount of the bacteria is in the range from 1×10³ to 1×10¹¹ colony forming units (CFU), more preferably in the range from 1×10⁵ to 1×10¹⁰ CFU.

In some embodiment, the bacterial compositions described herein are formulated for oral administration—e.g. in a cream, a gel, a paste or a rinse.

Contemplated compositions include, but are not limited to a toothpaste, tooth gel, tooth powder, tooth cleaning liquid, tooth cleaning foam, mouth wash, mouth spray, dental floss, chewing gum and lozenges.

According to a particular embodiment, the bacterial compositions are formulated into a chewing gum.

The composition according to embodiments of the invention may further comprise one or more components selected from the group consisting of carriers, excipients or further active ingredients such as, for example, active agents from the group of non-steroidal antiphlogistics, antibiotics, steroids, anti-TNF-alpha antibodies or other biotechnologically produced active agents and/or substances as well as analgetics, dexpanthenol, prednisolon, polyvidon iodide, chlorhexidine-bis-D-gluconate, hexetidine, benzydamine HCl, lidocaine, benzocaine, macrogol lauryl ether, benzocaine in combination with cetidyl pyridinium chloride or macrogol lauryl ether in combination with protein free hemodialysate from calf blood, as well as for example fillers (e.g. cellulose, calcium carbonate), plasticizer or flow improves (e.g. talcum, magnesium stearate), coatings (e.g. polyvinyl acetate phtalate, hydroxyl propyl methyl cellulose phtalate), disintegrants (e.g. starch, cross-linking polyvinyl pyrrolidone), softener (e.g. triethyl citrate, dibutyl phthalate) substances for granulation (lactose, gelatin), retardation (e.g. poly (meth)acrylic acid methyl/ethyl/2-trimethyl aminomethyl ester copolymerizates in dispersion, vinyl acetate/crotonic acid copolymerizates), compaction (e.g. microcrystalline cellulose, lactose), solvents, suspending or dispersing agents (e.g. water, ethanol), emulsifiers (e.g. cetyl alcohol, lecithin), substances for modifying the rheological properties (silica, sodium alginate), substances for microbial stabilization (e.g. benzalkonium chloride, potassium sorbate), preservatives and antioxidants (e.g. DL-alpha-tocopherol, ascorbic acid) substances for modifying pH (lactic acid, citric acid), blowing agents or inert gases (e.g. fluorinated chlorinated hydrocarbons, carbon dioxide), dyes (iron oxide, titanium oxide), basic ingredients for ointment (e.g. paraffines, bees wax) and others as described in the literature (e.g. in Schmidt, Christin. Wirk- und Hilfsstoffe fur Rezeptur, Defektur und Gro herstellung. 1999; Wissenschaftliche Verlagsgesellschaft mbH Stuttgart oder Bauer, Fromming Fuhrer. Lehrbuch der Pharmazeutischen Technologie. 8. Auflage, 2006. Wissenschaftliche Verlagsgesellschaft mbH Stuttgart).

Furthermore, the composition may be in the form of a solution, suspension, emulsion, tablets, granules, powder or capsules.

The compositions according to some embodiments of the present invention may contain abrasive systems (abrasive and/or polishing components) such as silicates, calcium carbonate, calcium phosphate, aluminum oxide and/or hydroxyl apatite, surfactants such as e.g. sodium lauryl sulfate, sodium lauryl sarcosinate and/or cocamidopropyl betaine, humectants such as glycerol and/or sorbitol, thickening agents, e.g. carboxy methyl cellulose, poly ethylene glycols, carrageenans and/or Laponite®, sweeteners such as saccharine, aroma and taste correcting agents for unpleasant taste impressions, taste modifying substances (e.g. inositol phosphate, nucleotides, e.g. guanosine monophosphate, adenosine monophosphate or other substances, e.g. sodium glutamate or 2-phenoxy propionic acid), cooling agents such as menthol derivates (e.g. L-mentyl lactate, L-menthyl alkyl carbonate, menthone ketals), icilin and icilin derivates, stabilizers and active agents such as sodium fluoride, sodium monofluoro phosphate, tin difluoride, quarternary ammonium fluorides, zinc citrate, zinc sulfate, tin pyrophosphate, tin dichloride, mixtures of different pyrophosphates, triclosane, cetyl pyridinium chloride, aluminum lactate, potassium citrate, potassium nitrate, potassium chloride, strontium chloride, hydrogen peroxide, aroma substances, sodium bicarbonate and/or smell correcting agents.

Chewing gums or dental care chewing gums may comprise a chewing gum base comprising elastomers, e.g. polyvinyl acetate (PVA), polyethylene, (low or medium molecular) polyiso butane (PIB), polybutadiene, isobutene/isoprene copolymers, polyvinyl ethyl ether (PVE), polyvinyl butyl ether, copolymers of vinyl esters and vinyl ethers, styrene/butadiene copolymers (SBR) or vinyl elastomers, e.g. based on vinyl acetate/vinyl laurate, vinyl acetate/vinyl stearate or ethylene/vinyl acetate and mixtures of the mentioned elastomers as e.g. example described EP 0 242 325, U.S. Pat. Nos. 4,518,615, 5,093,136, 5,266,336 5,601,858 or 6,986,709. Additionally chewing gum bases may contain further ingredients, e.g. (mineral) filers, e.g. calcium carbonate, titanium dioxide, silicone dioxide, talcum, aluminum oxide, dicalcium phosphate, tricalcium phosphate, magnesium hydroxide and mixtures thereof, plasticisers (e.g. lanolin, stearic acid, sodium stearate, ethyl acetate, diacetin (glycerol diacetate), triacetin (glycerol triacetate) and trietyhl citrate), emulsifiers (e.g. phosphatides, such as lecithin and mono and diglycerides of fatty acids, e.g. glycerol monostearate), antioxidants, waxes (e.g. paraffine waxes, candelilla waxes, carnauba waxes, microcrystalline waxes and polyethylene waxes), fats or fatty oils (e.g. hardened (hydrogenated) plant or animal fats) and mono, di or triglycerides.

Other examples of dental care products include, but are not limited to candy, lozenge, gelatin-gum, toffee, chewing gum, chew toy, biscuit, capsule, toothpaste, toothgel, prophylactic paste, toothpowder, mouthwash, mouthspray, solution, coated dental floss, coated interdental brush or coated toothbrush.

A “dental strip” or “dental strips” refer to one or more flexible substrates onto or into which are disposed one or more active agent(s) that are to be delivered to the surface(s) of one or more teeth.

In certain embodiments the dental strips are configured so that they adhere to the tooth surface and provide delivery of the active agent(s) over a period of time (e.g., 5 or 10 minutes up to 1, 2, 3, or 4 hours). In certain embodiments dental strips are fabricated to attach to and deliver one or more active agent(s) to the facial and/or lingual surfaces and/or occlusal surface(s) of the teeth. In certain embodiments one or more dental strips are designed to deliver active agent(s) to one or more surface(s) of incisors, and/or cuspids, and/or bicuspids, and/or molars. In various embodiments the dental strips contemplated herein include whitening agents.

The bacterial compositions may be administered via the oral cavity. The bacterial compositions may be useful for dental applications. For such applications they may be administered to the gums or teeth.

In some embodiments the compositions described herein are incorporated into a food product. The term “food product” as used herein refers to any substance containing nutrients that can be ingested by an organism to produce energy, promote health and wellness, stimulate growth, and maintain life. The term “enriched food product” as used herein refers to a food product that has been modified to include the composition comprising composition described herein, which provides a benefit such as a health/wellness-promoting and/or disease-preventing/mitigating/treating property beyond the basic function of supplying nutrients.

The probiotic composition can be incorporated into any food product. Exemplary food products include, but are not limited to, protein powder (meal shakes), baked goods (cakes, cookies, crackers, breads, scones and muffins), dairy-type products (including but not limited to cheese, yogurt, custards, rice pudding, mousses, ice cream, frozen yogurt, frozen custard), desserts (including, but not limited to, sherbet, sorbet, water-ices, granitas and frozen fruit purees), spreads/margarines, pasta products and other cereal products, meal replacement products, nutrition bars, trail mix, granola, beverages (including, but not limited to, smoothies, water or dairy beverages and soy-based beverages), and breakfast type cereal products such as oatmeal. For beverages, the probiotic composition described herein may be in solution, suspended, emulsified or present as a solid.

In one embodiment, the enriched food product is a meal replacement product. The term “meal replacement product” as used herein refers to an enriched food product that is intended to be eaten in place of a normal meal. Nutrition bars and beverages that are intended to constitute a meal replacement are types of meal replacement products. The term also includes products which are eaten as part of a meal replacement weight loss or weight control plan, for example snack products which are not intended to replace a whole meal by themselves, but which may be used with other such products to replace a meal or which are otherwise intended to be used in the plan. These latter products typically have a calorie content in the range of from 50-500 kilocalories per serving.

In another embodiment, the food product is a dietary supplement. The term “dietary supplement” as used herein refers to a substance taken by mouth that contains a “dietary ingredient” intended to supplement the diet. The term “dietary ingredients” includes, but is not limited to, the composition comprising the probiotic composition as described herein as well as vitamins, minerals, herbs or other botanicals, amino acids, and substances such as enzymes, organ tissues, glandulars, and metabolites.

In yet another embodiment, the food product is a medical food. The term “medical food” as used herein means a food which is formulated to be consumed or administered entirely under the supervision of a physician and which is intended for the specific dietary management of a disease or condition for which distinctive nutritional requirements, based on recognized scientific principles, are established by medical evaluation.

As used herein the term “about” refers to ±10%

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Materials and Methods

Strains and Growth Media

Cultures of the clinical isolate strain S. mutans UA159 were grown overnight in brain heart infusion broth (BHI, Acumedia, Landing, Mich.) at 37° C. in 95% air/5% CO₂. The following B. subtilis strains were used in this study: NCIB3610 (WT), and two B. subtilis strains, which were ordered from the Bacillus Genetic Stock Center (Ohio State University, USA) B. subtilis ΔgutB, B. subtilis ΔmltD. In order to transfer the mutation to the NCBI3610 strain, transduction using SPP1 phage was performed as described previously²⁸. To ensure that the mutant cells could not metabolise the alcoholic sugar, the cells were grown in M9 medium supplemented with sorbitol or mannitol and for control, the growth curves compared to WT as well as M9 containing glucose (FIGS. 6A-C). For starter culture generation, one colony of B. subtilis from fresh Lysogeny broth (LB, Neogen, Lansing, Mich., USA) agar plate was grown in LB and incubated at 37° C. at 150 rpm for 5 h. For all the experiments, both bacteria species were collected in late exponential phase.

All the experiments conducted in either TY (1.4% tryptone (Acumedia, Landing, Mich., USA), 0.8% yeast extract (Acumedia, Landing, Mich., USA)) ²⁹ medium or M9 minimal medium:

Mono and Dual-Species Biofilm Formation

For B. subtilis mono-species biofilm, a starter culture was diluted 1:100 (to obtain final O.D (600 nm)=0.07)) into TY or TY supplemented with either 2% sucrose or sorbitol or mannitol at various concentrations (10, 25,50,100 or 125 mM) in 96-well plate (Nunc, Roskilde, Denmark). The plate was incubated at 37° C. in 95% air/5% CO₂ for 24 h.

For S. mutans mono-species biofilm, overnight cultured S. mutans (O.D (600 nm)=1)) were diluted 1:10 into TY or TY supplemented with 2% sucrose (served as controls) or TY supplemented with different concentration of sorbitol or mannitol (10, 25,50,100 or 125 mM) into 96-well plate (Nunc). The plate was incubated at 37° C. in 95% air/5% CO₂ for 24 h. For dual-species biofilm, cells of B. subtilis and S. mutans were grown into 96-well plate as follows: to fresh TY or TY supplemented with 2% sucrose or different concentration of sorbitol or mannitol (10, 25, 50, 100 or 125 mM) were introduced overnight cultured S. mutans (diluted by a ratio 1:10, approximately 2.5×10⁷ CFU) and B. subtilis (diluted by a ratio 1:100, approximately 2.5×10⁵ CFU). The ratio between B. subtilis and S. mutans cells that were seeded to obtain the dual-species biofilm was approximately 1:100. The plate was incubated at 37° C. in 95% air/5% CO₂ for 24 h³⁰.

Quantification of Biofilm Biomass Using Crystal Violet Staining

The generated submerged biofilms were washed carefully twice with saline solution. The biofilms were stained using 0.1% Crystal Violet (CV) (Merck, Darmstadt, Germany) solution similarly as described previously^{31,32}. Following 20 min of incubation, the CV was washed twice with saline and the stained biofilms were dried overnight at RT. Next, 33% acetic acid was added to elute the CV for 30 min while shaking at RT. The extract was placed in a new 96-well plate and O.D. at 595 nm was measured using a plate reader instrument (infinite PRO2000, NEOTEC Scientific Instrumentation Ltd. Camspec, Cambridge, United Kingdom)^{31,32}.

Growth Curve Analysis

For growth curve analysis, B. subtilis or S. mutans cultures grown as described above, were diluted (1:100 or 1:10 respectively) in TY or TY supplemented with 2% sucrose or different concentration of sorbitol or mannitol. When needed, the bacteria were diluted in M9 medium with 0.2% glucose or 50 mM sorbitol/mannitol. The cultures were then incubated at 37° C. overnight with slight shaking. The growth was recorded every hour using optical density measurement at 595 nm (infinite PRO2000).

Gene Expression for Key Enzymes in the Sorbitol/Mannitol Metabolic Pathway

RNA Extraction

The cultures of either B. subtilis or S. mutans grown as described above, were diluted (1:100 or 1:10 respectively) in TY or TY supplemented with 50 mM sorbitol or mannitol. Next, B. subtilis were grown 37° C. at 150 rpm while S. mutans at 37° C. in 95% air/5% CO₂. Samples for each treatment were taken after 3, 6, 12, 18 and 24 hours.

One mL of RNA protect (Qiagen, Hilden, Germany) was added to each sample and incubated at RT for 5 min. After, the cells were centrifuged at 4,000×g for 10 min. RNA extraction preformed using GenUP total RNA kit (biotechrabbit, Hennigsdorf, Germany) according to the manufacture's protocol.

Reverse Transcription and Real-Time PCR

Reverse transcription was performed using qScript cDNA synthesis kit (Quantabio, Beverly, Mass., USA).The synthesized cDNA was later used for the real-time PCR analysis of key enzymes in the metabolic pathway of S. mutans and B. subtilis (Sorbitol dehydrogenase (gutB) or Mannitol-1-phosphate 5-dehydrogenase (mltD)). The RT-PCR reaction was performed as described previously³³. Briefly, RT-PCR reaction mixture (20 μL) contained 1×SYBR Green (Invitrogen, California, USA), 1 μL cDNA sample and 25 μM of the appropriate forward and reverse PCR primers. PCR conditions included an initial denaturation at 95° C. for 10 min, followed by a 40-cycles of amplification, consisting of denaturation at 95° C. for 15 s and annealing and extension at 60° C. for 1 min. Contamination by residual genomic DNA was determined from control reactions devoid of reverse transcriptase. The expression levels of all the tested genes by real-time PCR were normalized using the 16S rRNA gene of S. mutans as an internal standard (Shemesh et al., 2007) or sigA gene for B. subtilis.

Metabolic Activity

Microbial Metabolic Activity Using MTT Assay:

The assay measuring the cell proliferation rate was performed as described previously with some modifications. Briefly, B. subtilis or S. mutans cultures grown as described above, were diluted (1:100 or 1:10 respectively) in TY or TY supplemented with 50 mM sorbitol or mannitol. The bacterial cultures were grown at 37° C. in 95% air/5% CO₂. Samples for each treatment were taken following 3, 6, 9, 12, 18 and 24 hours of incubation into 96 well plate. To determine planktonic cell viability, 20 μl of the MTT solution (100 mM) was added to each well (in the multi-well plate). The plate was incubated for 1 h at 37° C. The MTT dissolved in 20 μl DMSO for 15 min while shaking at RT. absorbance values were measured at 570 nm³⁴.

Bacterial Respiration

The XFe 96Extracellular Flux Analyzer (Seahorse Bio-science) was used to quantitate oxygen consumption rates (OCRs). The starter cultures generated as described above, were diluted into fresh M9 media containing 50 mM or 100 mM sorbitol/mannitol and then 90 μL of diluted cells was added to XF Cell Culture Microplates pre-coated with poly-D-lysine (PDL). The Cells were incubated at 37° C. for 3 hours and then centrifuged for 10 min at 1,400×g to attach them to the pre-coated plates. After centrifugation, 90 μL of fresh M9 media was added containing 50 mM or 100 mM sorbitol/mannitol to each well. The measurements were taken for 3 h, every 15 min in 3 cycles reading following 3 min of shaking. Initial and final O.D were taken to monitor bacterial growth³⁵.

Secondary Metabolite Analysis

B. subtilis and S. mutans starters were diluted in TY supplemented with 50 mM sorbitol (1:100 or 1:10 accordingly) in mono-species cultures and were grown together in dual-species culture with the same cells ratio. All culture were incubated at 37° C. in 95% air/5% CO₂ for 6 h. 2 ml of each culture were centrifuge at 5000 rpm for 10 min and filtered using 0.45 μm filter (MillexGV, Merck). The samples were froze immediately in liquid nitrogen and lyophilized. All sample were transferred to the GCMS unit for preparation and analysis.

Statistical Analysis

The obtained data were statistically analysed using t-test. All tests applied were two-tailed, and a p-value of 5% or less was considered statistically significant. For the metabolomics assay PCA analysis was conducted. Statistical analysis was performed using Analyse-it add in tool for Microsoft Excel software.

Results

B. subtilis Mitigate Biofilm Formation by S. mutans in the Presence of Alcoholic Sugars

The initial phenotypic observations in this study indicated that B. subtilis eliminates biofilm formation by S. mutans in the presence of the alcoholic sugars such as sorbitol or mannitol. The results show that S. mutans could form enhanced biofilm (compared to the TY control) in the presence of either sorbitol or mannitol when cultured as a monospecies. Whereas, following introduction of B. subtilis to the system, the biofilm biomass of S. mutans was significantly reduced by more than 50% in all tested concentrations of the alcoholic sugars compare to the S. mutans mono-culture biofilm at the same concentration (FIGS. 1 and 7). However, in the presence of sucrose (a non-alcoholic sugar) there was no effect of B. subtilis cells on the S. mutans biofilm biomass in the mixed culture. As expected, B. subtilis mono-culture did not adhere to the surface and could not form a submerge biofilm (FIG. 7).

B. subtilis is More Compatible to Grow in the Presence of Alcoholic Sugars

As a next step, the present inventors examined the growth of the tested bacterial species. Growth curve analysis indicated that B. subtilis cells could actively enter the log phase, whereas the cells of S. mutans required relatively prolonged lag phase in the presence of sorbitol (FIG. 2A) and mannitol (FIG. 2B) for approximately 3 hours. It was also remarkable that an addition of sorbitol (FIG. 2A) or mannitol (FIG. 2B) (at different concentrations) resulted in a small increase in the final O.D of the tested bacterial cells following 16 h of growth (FIGS. 2A and 2B).

Since TY medium could be considered as a rich nutrient medium, bacterial growth in M9 medium (a minimal medium for which the carbon source and amount can be controlled) was analyzed (FIG. 2C). Growth curve analysis confirmed that the cells of B. subtilis are capable of growing in the presence of either sorbitol or mannitol even in a minimal medium (FIG. 3). Moreover, it could be observed that B. subtilis growth requires addition of a carbon source into M9 medium (FIG. 2C). Conversely, the cells of S. mutans could grow only in the presence of glucose but not with sorbitol or mannitol as a sole carbon source for 16 hours of growth.

Enrichment of Growth Media with Alcoholic Sugars Up-Regulates Genes Encoding for Key Enzymes in Cellular Metabolism

Differences between the abilities of Bacillus and Streptococcus cells to grow in the presence of different sugars raised the question about regulation of key enzymes involved in either sorbitol or mannitol metabolism. Therefore, the present inventors tracked the regulation profile of genes encoding for sorbitol dehydrogenase (gutB) and mannitol 1 phosphate dehydrogenase (mltD) (FIG. 3). The effect on gene expression was compared to the same growth time point in TY alone, or TY supplemented alcoholic sugars. Both genes, gutB and mltD, were up-regulated in the presence of either sorbitol or mannitol compare to TY medium alone (the expression value in TY control was adjusted to 1). The induction occurred in the early stages after the addition of the sugar to the medium. After 3 hours it reached the maximal induction rate of 2 folds for gutB and 1.6 folds for mltd (FIG. 3). After 6 hours and for the rest of the time point that were checked until 24 hours, the induction was similar and stable at 1.6 fold of induction for gutB and 1.2 fold induction for mltD (FIG. 3). On the other end, the induction pattern in both of the genes in S. mutans was different. Although we could see up-regulation in both of the genes after 3 hours of incubation (FIG. 3), the maximal up-regulation occur after 18 hours of incubation with the sugars and then a small decrease in the induction folds (FIG. 3). These results indicate that there is a divergence in the regulation profiles of the key metabolism enzymes of the tested bacteria.

B. subtilis Cells Metabolize Alcoholic Sugars More Effectively Compared to S. mutans Cells

Since it was observed that S. mutans cells could not grow in the presence of alcoholic sugars in minimal medium for 16 hours, but an up-regulation in the expression of genes for metabolic enzymes was detected, it was hypothesized that although there is transcription of the gene, there might be a delay in the translation and therefore a low metabolic activity in S. mutans. To test this hypothesis, the present inventors estimated the metabolic activity of the bacteria using MTT assay. The metabolic activity of B. subtilis and S. mutans increased during the 9 hours of incubation in the presence of the sugars and did not show a significant difference between the bacteria (FIG. 4A). However, MTT is not only reflecting the metabolic activity of the bacteria but can also be influenced by the number of viable cells³⁶.

Therefore, a bacterial respiratory assay that measures in real-time the oxygen consumption rate (OCR) of the bacteria as an indication for the metabolic activity was performed (FIG. 4B). Interestingly, while the MTT results did not designate a significant difference between the bacteria, in the respiratory assay a notable difference between the metabolic stages of the bacteria was detected (FIG. 4B). During the 3 hours of incubation (hours 3-6 of incubation from the starting point of the experiment and introduction to the media with the sugars), the OCR of B. subtilis was much higher than the OCR of S. mutans (FIG. 4B). Moreover, the OCR of B. subtilis in the presence of sorbitol (FIG. 4B) was higher than the OCR of B. subtilis in the presence of mannitol (FIG. 4B). Thus, the present results indicate that the metabolic activity of B. subtilis in the presences of alcoholic sugars is higher than the metabolic activity of S. mutans and in addition, its metabolic activity is higher in the presence of sorbitol compare to mannitol. Importantly, the lack in OCR in S. mutans is not a result of dead or not growing bacteria. CFU counts in three central time points during the experiment shows that S. mutans cells are alive and dividing although to a much lesser degree compared to B. subtilis cells that started less and grew extensively (FIG. 8).

Lastly, in order to assess the metabolic activity of B. subtilis and S. mutans in the mixed culture in the presence of sorbitol, we performed a secondary metabolic analysis using GCMS to the growth media of each bacterium alone and to the media of the dual-species culture. To compare the identified secondary metabolites between the three groups, PCA analysis was conducted³⁷. The PCA analysis can explain up to 65% of the variation between the three groups showing that the metabolites profile of B. subtilis and the mixed culture is more similar than the metabolite profile of S. mutans mono-culture. Although the results suggest that B. subtilis is more active metabolically in the dual culture, it seems that there is still metabolic activity by S. mutans cells in the dual-species culture.

Metabolic Effectiveness of B. subtilis Cells at Early Stages of Growth has an Important Role in Mitigating Biofilm Formation

According to the results described above, it is conceivable that B. subtilis is more compatible to utilize the alcoholic sugars due to its early expression in key enzymes: sorbitol dehydrogenase and mannitol 1 phosphate dehydrogenase (encoded by gutB and mltd, respectively). To prove this notation, the ability of ΔgutB and ΔmltD knock out mutants of B. subtilis was tested in mitigating biofilm formation by S. mutans in the presence either sorbitol or mannitol. The inhibitory effect of B. subtilis cells on biofilm formation by S. mutans was notably reduced in the presence of either sorbitol or most of the concentrations of mannitol (FIGS. 5A-B). Interestingly, the deletion mutation did not affect only the related sugar but also had an effect in the presence of the other sugar in high concentrations, where the reduction in biofilm biomass in the dual-species culture was lower or absent (ΔgutB in the presence of mannitol or ΔmltD in the presence of sorbitol) (FIGS. 5A-B).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A composition comprising at least one sugar alcohol and a population of viable probiotic bacteria belonging to the class Bacilli, wherein the product is devoid of sugar or comprises no more than 5% of the amount of said sugar alcohol in the composition, the composition being formulated for oral delivery.

2. The composition of claim 1, wherein said probiotic bacteria belong to the genus Bacillus.

3. The composition of claim 1, being a dental product.

4. A food comprising the composition of claim 1.

5. A device which is coated with the composition of claim 1.

6. The device of claim 5, being a toothpick or a dental floss.

7. The composition of claim 3, being selected from the group consisting of a wash, a paste, a chewing gum and an ointment.

8. The food of claim 4, further comprising an artificial sweetener.

9. The composition of claim 1, wherein said probiotic bacteria belong to the species Bacillus subtilus.

10. The composition of claim 1, wherein said sugar alcohol is a six carbon sugar alcohol.

11. The composition of claim 10, wherein said six carbon sugar alcohol is selected from the group consisting of mannitol, sorbitol, galactitol, flucitol and iditol.

12. The composition of claim 1, wherein said sugar alcohol is mannitol or sorbitol.

13. A method of preventing or treating dental caries in a subject comprising administering to the subject a therapeutically effective amount of a population of viable probiotic bacteria belonging to the class Bacillus and at least one sugar alcohol, thereby preventing or treating dental caries.

14. The method of claim 13, wherein said probiotic bacteria belong to the genus Bacillus.

15. The method of claim 13, wherein said viable probiotic bacteria and said at least one sugar alcohol are co-formulated in a single composition.

16. The method of claim 13, wherein said viable probiotic bacteria and said at least one sugar alcohol are formulated in separate compositions.

17. A method of preventing or treating dental caries in a subject comprising applying the composition of claim 3 to the oral cavity of a subject in need thereof, thereby preventing or treating dental caries in the subject.

18. The method of claim 13, wherein said probiotic bacteria belong to the species Bacillus subtilus.

19. The method of claim 13, wherein said sugar alcohol is a six carbon sugar alcohol.

20. The method of claim 19, wherein said six carbon sugar alcohol is selected from the group consisting of mannitol, sorbitol, galactitol, flucitol and iditol.

21. The method of claim 13, wherein said sugar alcohol is mannitol or sorbitol.

22. The method of claim 17, wherein said probiotic bacteria belong to the species Bacillus subtilus.

23. The method of claim 17, wherein said sugar alcohol is a six carbon sugar alcohol.

24. The method of claim 23, wherein said six carbon sugar alcohol is selected from the group consisting of mannitol, sorbitol, galactitol, flucitol and iditol.

25. The method of claim 17, wherein said sugar alcohol is mannitol or sorbitol.
